(19) United States
(12) Patent Application Publication
(10) Pub. No.: US 2012/0028363 A1
(43) Pub. Date: Feb. 2, 2012

(54) METAL POROUS MATERIAL, METHOD FOR PREPARING THE SAME AND METHOD FOR DETECTING NITROGEN-CONTAINING COMPOUNDS

(75) Inventors: Chun-Nan Kuo, Taichung County (TW); Shou-Nan Li, Nantou County (TW); Shaw-I Yen, Hsinchu County (TW); Yu-Lun Lai, Tainan County (TW); Jung-Nan Hsu, Taichung City (TW)

(73) Assignee: INDUSTRIAL TECHNOLOGY RESEARCH INSTITUTE, Hsinchu County (TW)

(21) Appl. No.: 13/112,382

(22) Filed: May 20, 2011

(30) Foreign Application Priority Data
Aug. 2, 2010 (TW) 099125578

(51) Int. Cl. G01N 33/00

(52) U.S. Cl. 436/113; 436/106

(57) ABSTRACT

The invention provides a metal porous material, a method for preparing the same, and a method for detecting nitrogen-containing compounds. The method for fabricating metal porous material includes: mixing a siloxane, a metal or metallic compound, and water, to obtain a mixture after stirring; modifying the mixture to a pH value of less than 7; subjecting the mixture to a first dry treatment to obtain a solid; after polishing the solid to obtain a powder, subjecting the powder to a second dry treatment. It should be noted that the method is free of any annealing or calcination process.

### CROSS REFERENCE TO RELATED APPLICATIONS

This application is based upon and claims the benefit of priority from the prior Taiwan Patent Application No. 099125578, filed on Aug. 2, 2010, the entire contents of which are incorporated herein by reference.

### BACKGROUND

1. Field of the Invention

This application relates to a metal porous material, and in particular relates to a metal porous material serving as detecting material for a gas detector.

2. Description of the Related Art

Monitoring and controlling micro contaminants, is one of the most important issues for IC manufactures, as critical dimensions continue to shrink.

International Technology Roadmap for Semiconductor (ITRS) predicts that the critical dimensions of a chip scale will shrink to 32 nm in 2013. Thus, controlling micro contaminants is critical for IC manufacturers. For example, for 32 nm semiconductor processes, a recommended sensitive area micro contaminants (such as acid, base, organic compounds or dopants) value for a clean room is less than 10 ppt to 150 ppt. Therefore, a gas sensor having a low detection limit is needed, to assure that the air quality in a clean room meets advanced semiconductor process requirements.

In the semiconductor wafer processing, the concentration of ammonia (NH₃) should be detected and controlled on a parts-per-billion scale. In lithography processes, even low concentrations of airborne molecular contaminates can reduce device yields and increase the incidence of defects. For example, concentrations of ammonia at part-per-billion (ppb) levels can react with photoresists and lead to “T-topping”. Further, ammonia is a photo-reactive gas and may react with sulfide (such as SO₂) disposed on the lens (employed by the photolithography device) to obtain (NH₄)₂SO₂ which blurs the surface of lens, resulting in damaging the process equipments.

In a semiconductor factory, the ammonia contamination sources includes a CVD process, a wafer cleaning process, a photoresist coating process, a chemical mechanical polishing process, and gases exhaled by humans. Although there are air circulation systems with various filtration functions employed in the clean room and/or process equipments to ensure an appropriate atmosphere, it is still necessary to provide a highly sensitive nitrogen-containing compound sensor for providing real-time notification of contamination concentrations, thereby ensuring the maintenance of manufacturing yields.

The conventional ammonia sensor, which has a detection limit of about between 1 ppm and 1 sub-ppm, cannot meet the demands of a semiconductor factory. In order to reach the detection limit for detecting parts-per-billion scale ammonia, the techniques, utilized within the sensors for detecting ammonia, employed by the semiconductor factory includes ion mobility spectroscopy (IMS) techniques, chemiluminescence techniques, cavity ring-down spectroscopy (CRDS) techniques, and impinger with ion chromatography techniques. However, a lot of time, labor, materials and/or expensive analytical instrumentations are required for the aforementioned techniques, and real-time detection is not accomplished, thereby lowering fabrication yields.

Accordingly, a novel material and technique for detecting ammonia is desired to address the described problems.

### SUMMARY

An exemplary embodiment of a method for fabricating metal porous material includes the following steps: mixing a siloxane, a metal or metallic compound, and water, to obtain a mixture after stirring; modifying the mixture to a pH value of less than 7; subjecting the mixture to a first dry treatment to obtain a solid; and polishing the solid to obtain a powder, wherein the powder is subjected to a second dry treatment, wherein the method for fabricating metal porous material is free of any annealing or calcination process.

The disclosure also provides a metal porous material including a product fabricated by the aforementioned method. An exemplary embodiment of a metal porous material includes: at least one metal element selected from the group consisting of Fe, Cu, V, Mn, Cr, Co, and combinations thereof, wherein the atomic ratio of the metal element to the metal porous material is between 1-10%; a silicon element, wherein the atomic ratio of the silicon element to the metal porous material is between 20-40%; and an oxide element, wherein the atomic ratio of the silicon element to the metal porous material is between 50-70%, wherein the metal porous material has a decomposition point of between 150-250° C.

The disclosure also provides a method for detecting nitrogen-containing compounds including the following steps: providing the aforementioned metal porous material; introducing a gas sample to react with the metal porous material; and analyzing results of the reaction.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a flow chart of a method for fabricating the metal porous material according to an embodiment of the disclosure.

FIG. 2 is a schematic view illustrating a detector for detecting nitrogen-containing compounds according to Example 9 of the disclosure.

FIG. 3 shows a graph plotting the absorption intensity against the wavelength of the metal porous material of Example 9.

FIG. 4 shows a graph plotting the absorption intensity variation (ΔA) against the wavelength under various concentrations of NH₃.

FIG. 5 shows the results of Example 15 for estimating the reusability of the metal porous material.

### DETAILED DESCRIPTION

The following description is of the best-contemplated mode of carrying out the invention. This description is made for the purpose of illustrating the general principles of the invention and should not be taken in a limiting sense. The scope of the invention is best determined by reference to the appended claims.

The disclosure provides a metal porous material which shows visible color change after absorbing nitrogen-containing compounds. Particularly, the metal or metallic compound performs a gradient color conversion from the original color to a specific color after reacting with the nitrogen-containing compounds. Due to the specific fabricating method of the metal porous material, the metal or metallic compound stably exists within the porous siloxane, thereby providing a sufficient space to the metal or the metal compound for reacting with the target compound (such as ammonia) and improving the detection limit.

The disclosure also provides a method for detecting nitrogen-containing compounds. In combination with a UV-Visible spectroscopy system, the method can quantize the absorption intensity of the metal porous material, and a near-linear relationship between the concentration of the absorbed nitrogen-containing compound and the absorption intensity variation (ΔA) can be built. Therefore, the concentration of a unknown nitrogen-containing atmosphere can be determined by means of the method of the disclosure.

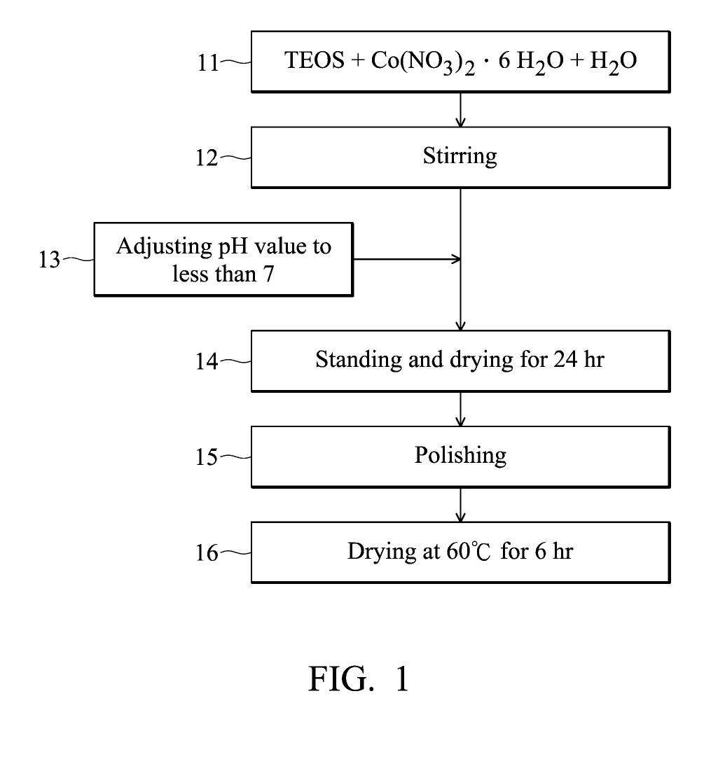

In an embodiment of the disclosure, the metal porous material can be prepared by the following steps. FIG. 1 shows a flow chart of a method for fabricating the metal porous material. First, siloxane, metal (or metallic compound), and water are mixed (step 11). After stirring (step 12), a mixture is obtained. Next, the pH value of the mixture is adjusted to be less than 7.0 (step 13). After adjusting the pH value, the mixture is left standing and is subjected to a first dry treatment at a specific temperature (such as room temperature) for a period of time (such as 24 hrs) to obtain a solid (step 14). Next, the solid is polished to obtain a powder (step 15), and the powder is subjected to a second dry treatment at a specific temperature (such as 60° C.) (step 16), obtaining the metal porous material. Particularly, the first and second dry treatment of the disclosure are both performed under a temperature of not more than 60° C. Further, the method for fabricating metal porous material is free of any annealing or calcination process (the process temperature during fabrication of the metal porous material is not more than 60° C.).

The metal porous material of the disclosure consists of at least one metal element (selected from the group consisting of Fe, Cu, V, Mn, Cr, Co, and combinations thereof and derived from metal or metallic compound) with an atomic ratio of between 1-10% (based on the total atomic amount of the metal porous material); silicon elements (derived from siloxane) with an atomic ratio of between 20-40% (based on the total atomic amount of the metal porous material); and oxide elements with an atomic ratio of between 50-70% (based on the total atomic amount of the metal porous material). It should be noted that, since the method for fabricating the metal porous material is free of any annealing or calcination process, the metal porous material has a decomposition point of between 150-250° C. To the contrary, a metal oxide fabricated through an annealing or calcination process has a decomposition point of more than 300° C.

Herein, the siloxane can have a chemical structure represented by Si(OR4), wherein R is C1-8 alkyl group. For example, the siloxane can be titanium (IV) isopropoxide (TTIP), tetramethoxysilane (TMOS), tetraethoxysilane (TEOS), or combinations thereof. The metal includes Fe, Cu, V, Mn, Cr, Co, or combinations thereof. Further, the metal compound includes halide of Fe, Cu, V, Mn, Cr, or Co, sulfide of Fe, Cu, V, Mn, Cr, or Co, nitrate of Fe, Cu, V, Mn, Cr, or Co, phosphate of Fe, Cu, V, Mn, Cr, or Co, sulfate of Fe, Cu, V, Mn, Cr, or Co, or combinations thereof, such as ferric nitrate, cobalt nitrate, chromium nitrate, or compounds having crystal water thereof.

The metal porous material has a silicon element/metal element weight ratio of between 0.95:0.05 and 0.05:0.95. When the metal element has a weight ratio of more than 0.95 (based on the total weight of the silicon element and the metal element), the metal porous material is apt to aggregate and have a large grain size; thereby reducing the active region surface area and reaction activity. On the other hand, when the metal element has a weight ratio of less 0.05 (based on the total weight of the silicon element and the metal element), the metal porous material has a relatively low active region surface area, resulting in reduced reaction activity.

An acid can be added to adjust the pH value of the solution. In one embodiment, the acid includes hydrochloric acid, sulfuric acid, phosphorus acid, nitric acid or combinations thereof. For instance, when the added metal salt is copper (II) chloride, hydrochloric acid is preferably used to adjust the pH value of the solution. The pH value of the solution is between about 7.0 and about 1.0, preferably between about 5.0 and about 2.0, for promoting the subsequent combination of metal (of the metal porous material) and ammonia (a basic compound).

According to another embodiment of the disclosure, a method for detecting nitrogen-containing compounds employing the aforementioned metal porous material is provided. The method includes providing the aforementioned metal porous material, introducing a gas sample to react with the metal porous material, and analyzing results of the reaction. The nitrogen-containing compounds include ammonia gas (NH₃)

In comparison with a conventional method for detecting nitrogen-containing compounds, the method of the invention employs metal porous materials having high selectivity for nitrogen-containing compounds. The metal porous materials may be further used as a sensor for a nitrogen-containing compound detector. The sensor would have a detection limit of less than about 100 ppt.

In one further embodiment, the sensor may be connected to an ultraviolet-visible spectroscopy system to form a real-time nitrogen-containing compound detector.

The method for real-time detection of nitrogen-containing compounds includes the following steps. A gas sample and a carrier gas such as nitrogen or noble gases respectively pass through different mass flow controllers and mixed together. The mixed gas is introduced to pass through the metal porous material, and then is exhausted. It should be noted that, since the absorption intensity detected by the ultraviolet-visible spectroscopy system within a specific wavelength range (such as 300-900 nm) is in direct proportion to the nitrogen-containing compound concentration adsorbed by the metal porous material, the nitrogen-containing compound concentration of the gas sample can be identified via the absorption variation of the metal porous material.

The following examples are intended to illustrate the invention more fully without limiting their scope, since numerous modifications and variations will be apparent to those skilled in this art.

### Example 1

First, 0.4 g of Co(NO₃)₂.6H₂O, and 8 ml of TEOS were mixed and added into 4 ml of water, obtaining a mixture. Next, 2 ml of HCl (2M) was added into the mixture, obtaining a solution with a pH value of less than 7. Next, the solution was left standing at room temperature for 24 hrs. After drying at room temperature, the obtained solid was subjected to a polishing process, obtaining a powder. Finally, the powder was subjected to a drying process with a temperature of 60° C. for 6 hrs, obtaining a cobalt and silicon containing porous material 1.

The surface of the cobalt and a silicon-containing porous material 1 was analyzed by an energy dispersive X-ray (EDX) spectrometer. The results of the measurements show that the ratio between the cobalt and the silicon of the nanostructure material was 12:88.

### Example 2

First, 0.4 g of Co(NO₃)₂.6H₂O, and 8 ml of TEOS were mixed and added into 4 ml of water, obtaining a mixture. Next, 0.12 ml of HCl (0.1M) was added into the mixture, obtaining a solution with a pH value of less than 7. Next, the solution was left standing at room temperature for 24 hrs. After drying at room temperature, the obtained solid was subjected to a polishing process, obtaining a powder. Finally, the powder was subjected to a drying process with a temperature of 60° C. for 6 hrs, obtaining a cobalt and silicon containing porous material 2.

### Example 3

First, 0.8 g of Co(NO₃)₂.6H₂O, and 8 ml of TEOS were mixed and added into 4 ml of water, obtaining a mixture. Next, 0.12 ml of HCl (0.1M) was added into the mixture, obtaining a solution with a pH value of less than 7. Next, the solution was left standing at room temperature for 24 hrs. After drying at room temperature, the obtained solid was subjected to a polishing process, obtaining a powder. Finally, the powder was subjected to a drying process with a temperature of 60° C. for 6 hrs, obtaining a cobalt and silicon containing porous material 3.

### Examples 4-8

Similar processes to that according to Example 1 were performed for Examples 4-8 except that Co(NO₃)₂6H₂O was replace with various metallic compounds. The employed metallic compounds of Examples 4-8 are shown in Table 1.

TABLE 1

| Example No. | metallic compound |
|---|---|
| 4 | Fe(NO₃)₃•9 H₂O |
| 5 | Cu(NO₃)₂•6 H₂O |
| 6 | VOSO₄•xH₂O(x > 1) |
| 7 | Mn(NO₃)₂•4H₂O |
| 8 | Cr(NO₃)₂•9 H₂O |

### Example 9

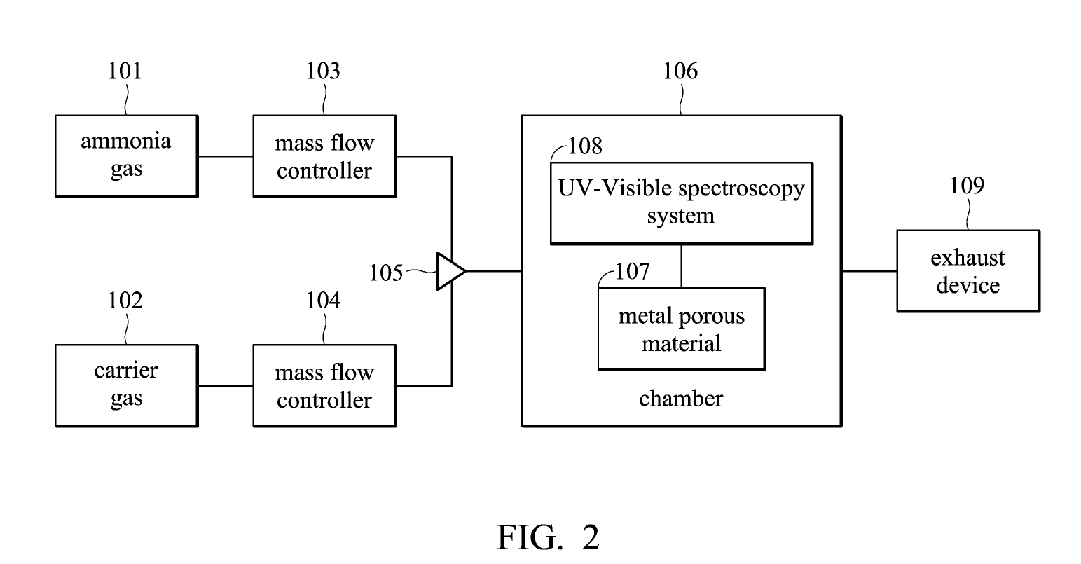

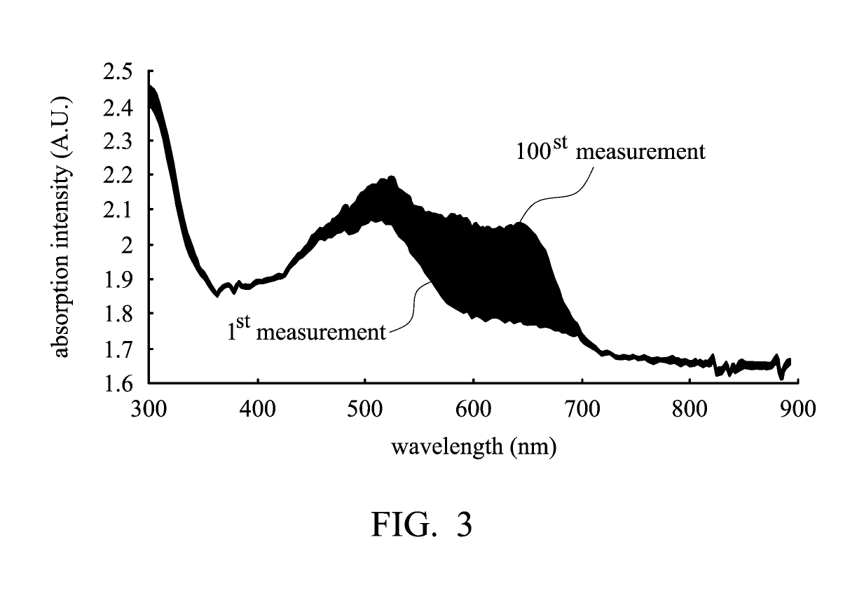

A method including the following steps was used to estimate the absorption efficiency of the cobalt and the silicon containing porous material 1. First, the cobalt and silicon containing porous material 1 prepared by Example 1 were located in the chamber 106 as shown in FIG. 2. An ammonia gas 101 and a carrier gas 102 (nitrogen gas) were respectively passed through different mass flow controllers 103, and 104 and mixed together, obtaining a mixed gas sample (with a NH3 concentration of 500 ppb). The valve 105 was used to control the mixed gas introduced to the chamber 106 with the metal porous material 107 therein (with a flow of 1700 sccm). It should be noted that the mixed gas was introduced to pass through the metal porous material 107 and then was exhausted by an exhaust device 109. A UV-Visible spectroscopy system 108 was used to measure the UV-Visible absorption spectrum of the metal porous material 107 per 2.5 minute for 250 minutes (at a temperature of 21.3° C. and a relative humidity of 44.1%), and the results are shown in FIG. 3. During measurement, the color of the metal porous material 107 gradually changed from pink to purple blue. Referring to FIG. 3, the absorption intensity between 600-700 nm wavelength was proportional to the introduced NH₃ gas volume. Therefore, the metal porous material can serve as a colorimetric detecting material for NH₃. Further, a detector employing the metal porous material of the invention can be connected to a UV-Visible spectroscopy system to form a real-time gas detector.

### Examples 10-11

For Examples 10-11, similar processes with that according to Example 9 were performed, except that the metal porous material prepared by Example 1 was replaced with the metal porous materials prepared by Examples 2 and 3. The employed metallic compounds and the absorption intensity variation (ΔA) (measured at a wavelength of 640 nm) of the metal porous materials of Examples 9-11 are shown in Table 2.

TABLE 2

| | | | | | absorption |
|---|---|---|---|---|---|
| | | | sampling | | intensity |
| | | concentration | frequency | Flow rate | variation |
| Example | metal porous material | of NH₃ (ppb) | (min)/times | (sccm) | (ΔA) |
| 9 | metal porous material | 500 | 2.5/100 | 1730 | 0.098 |
| | prepared by Example 1 | | | | |
| | (Co(NO₃)₂•6 H₂O:0.4 g); | | | | |
| | 2MHC:2 ml) | | | | |
| 10 | metal porous material | 500 | 2.5/100 | 1730 | 0.160 |
| | prepared by Example 2 | | | | |
| | (Co(NO₃)₂•6 H₂O:0.4 g); | | | | |
| | 0.1MHCl:0.12 ml | | | | |
| 11 | metal porous material | 500 | 2.5/100 | 1730 | 0.200 |
| | prepared by Example 3 | | | | |
| | (Co(NO₃)₂•6 H₂O:0.8 g); | | | | |
| | 0.1MHCl:0.12 ml | | | | |

### Examples 12-14

For Examples 12-14, similar processes with that according to Example 9 were performed, except that the concentration of NH₃ (500 ppb) was replaced with 60 ppb, 115 ppb, and 230 ppb respectively. The employed metallic compounds and the absorption intensity variation (ΔA) (measured at a wavelength of 640 nm) of the metal porous materials of Examples 12-14 are shown in Table 3.

TABLE 3

| | | concen- | sampling | | absorption |
|---|---|---|---|---|---|
| | | tration | frequency | flow | intensity |
| | metal porous | of NH₃ | (min)/ | rate | variation |
| Example | material | (ppb) | times | (sccm) | (ΔA) |
| 12 | metal porous | 60 | 2.5/100 | 1730 | 0.002 |
| | material | | | | |
| | prepared by | | | | |
| | Example 1 | | | | |
| 13 | metal porous | 115 | 2.5/100 | 1730 | 0.027 |
| | material | | | | |
| | prepared by | | | | |
| | Example 1 | | | | |
| 14 | metal porous | 230 | 2.5/100 | 1730 | 0.074 |
| | material | | | | |
| | prepared by | | | | |
| | Example 1 | | | | |
| 9 | metal porous | 500 | 2.5/100 | 1730 | 0.199 |
| | material | | | | |
| | prepared by | | | | |
| | Example 1 | | | | |

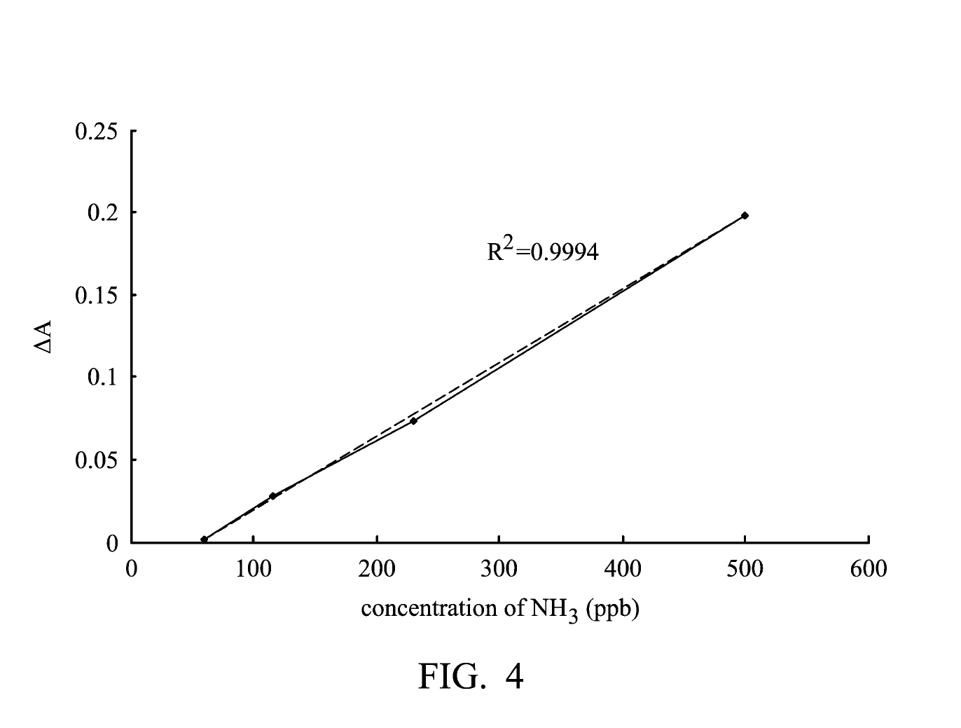

FIG. 4 shows a graph plotting concentration of NH3 against absorption intensity variation (ΔA) according to Table 3. As shown in FIG. 4, the concentration of NH3 is in direct proportion to the absorption intensity variation, indicating a near-linear relationship therebetween. Therefore, the metal porous material of the disclosure is not only applicable to qualitative analysis of ammonia gas, and but it is also applicable to quantitative analysis of ammonia gas in combination with the UV-Visible spectroscopy system.

### Example 15

The cobalt and silicon containing porous material 1 prepared by Example 1 was located in a chamber. A UV-Visible spectroscopy system was used to measure the UV-Visible absorption spectrum of the metal porous material before introducing a gas sample.

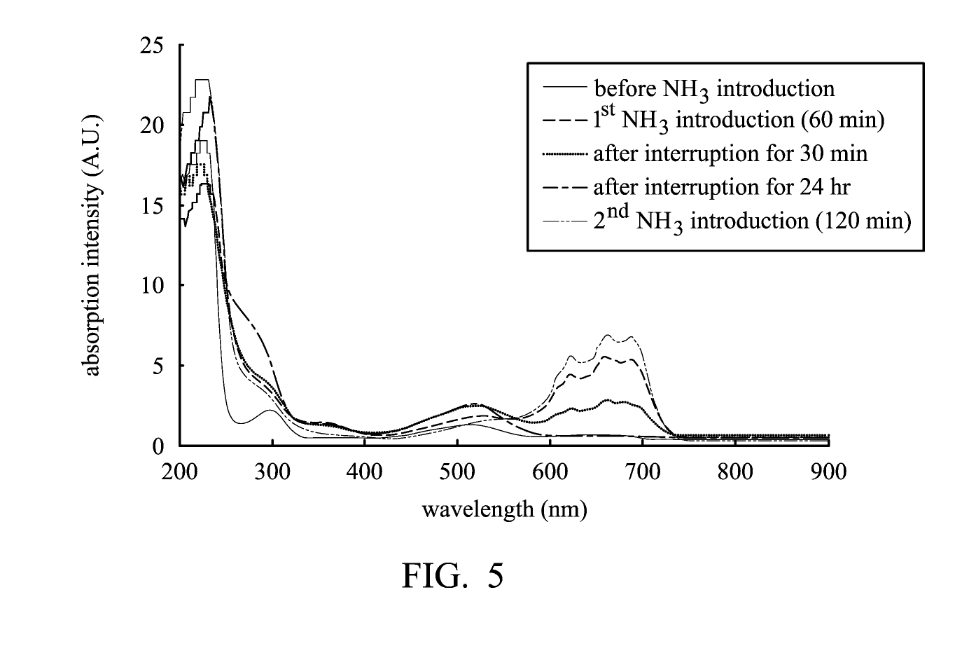

Next, a gas sample (with a NH₃ concentration of 46 ppm, 50 sccm) was introduced into the chamber having porous material for 60 min. Next, the UV-Visible absorption spectrum of the metal porous material was measured. Next, the introduction of the gas sample was interrupted. After 30 min, the UV-Visible absorption spectrum of the metal porous material was measured. Next, after 24 hrs, the UV-Visible absorption spectrum of the metal porous material was measured. Finally, the gas sample (with a NH3 concentration of 46 ppm, 50 sccm) was introduced again into the chamber having porous material for 120 min, and the UV-Visible absorption spectrum of the metal porous material was measured. The results are shown in FIG. 5. As shown in FIG. 5, the metal porous material of the disclosure exhibits excellent reusability and is suitable for detecting nitrogen-containing compounds.

Accordingly, since the positively charged metal of the metal porous material can be further bonded to the nitrogen with lone-pair electrons to produce transition metal compounds enhancing the absorption intensity within the visible spectroscopy, the metal porous material can serve as detecting material and can further combine with a UV-Visible spectroscopy system for qualitative and quantitative analysis of nitrogen-containing compounds. Moreover, the sensor employing the metal porous material of the disclosure has advantages of high sensitivity, high selectivity, excellent reusability, and low detection limit for detecting nitrogen-containing compound, and is suitable for detecting nitrogen-containing compound with low concentration.

Table 4 shows the comparison between the method for detecting nitrogen-containing compounds of the disclosure, Ion Mobility Spectroscopy (IMS), Chemiluminescence, Cavity Ring-Down Spectroscopy (CRDS), and Impinger with Ion chromatography.

TABLE 4

| | | Ion | | Cavity | |
|---|---|---|---|---|---|
| | The sensor | Mobility | Chemilumi- | Ring-Down | Impinger + ion |
| | of the | Spectros- | nescence | Spectros- | chromatography |
| | disclosure | copy (IMS) | (CI) | copy (CRDS) | (Impinger + IC) |
| response | 30 min | 30-60 min | 30-60 min | 20-30 min | 2-15 hr |
| time | | | | | |
| object | nitrogen- | NMP and | nitrogen- | NH₃ | MB |
| | containing | NH₃ | containing | | |
| | compound | | compound | | |
| detection | 100 ppt | 500 ppt | 500 ppt | 100 ppt | 100 ppt |
| limit | | | | | |
| interference | low | under the | low | under the | under the |
| | | influence of | | influence of | influence of |
| | | fluctuating | | fluctuating | ammonium |
| | | temperature | | relative | salt) |
| | | and | | humidity | |
| | | relative | | | |
| | | humidity | | | |
| additional | — | radiation | ozone genera- | — | ion chromato- |
| equipment | | emitting | tor, vacuum | | graphy |
| | | source | pump | | device |
| detecting | absorption | measuring | fluorescence | measuring | Impinging |
| principle | intensity | the ionic | | the | and Ion |
| | variation | molecule | | absorption | Chromato- |
| | (measuring | | | spectrum of | graphy by |
| | the bonding | | | a molecule | |
| | between the | | | excited | |
| | metal | | | laser | |
| | compound | | | | |
| | and the test | | | | |
| | sample) | | | | |

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A method for fabricating metal porous material, comprising:
mixing a siloxane, a metal or metallic compound, and water, to obtain a mixture after stirring;
modifying the mixture to a pH value of less than 7;
subjecting the mixture to a first dry treatment to obtain a solid; and
polishing the solid to obtain a powder, wherein the powder is subjected to a second dry treatment,
wherein the method for fabricating metal porous material is free of any annealing or calcination process.

2. The method as claimed in claim 1, wherein the siloxane has a structure represented by Si(OR)₄, wherein R is C₁₋₈ g alkyl group.

3. The method as claimed in claim 1, wherein the siloxane is titanium (IV) isopropoxide (TTIP), tetramethoxysilane (TMOS), tetraethoxysilane (TEOS), or combinations thereof.

4. The method as claimed in claim 1, wherein the metal comprises Fe, Cu, V, Mn, Cr, Co, or combinations thereof.

5. The method as claimed in claim 1, wherein the metal compound comprises halide of Fe, Cu, V, Mn, Cr, or Co, sulfide of Fe, Cu, V, Mn, Cr, or Co, nitrate of Fe, Cu, V, Mn, Cr, or Co, phosphate of Fe, Cu, V, Mn, Cr, or Co, sulfate of Fe, Cu, V, Mn, Cr, or Co, or combinations thereof.

6. The method as claimed in claim 1, wherein the metal porous material has a silicon element/metal element weight ratio of between 0.95:0.05 and 0.05:0.95.

7. The method as claimed in claim 1, wherein the process temperatures of the first dry treatment and the second dry treatment are both less than 60° C.

8. A metal porous material, consisting of:
at least one metal element selected from the group consisting of Fe, Cu, V, Mn, Cr, Co, and combinations thereof, wherein the atomic ratio of the metal element to the metal porous material is between 1-10%;
a silicon element, wherein the atomic ratio of the silicon element to the metal porous material is between 20-40%; and
an oxide element, wherein the atomic ratio of the silicon element to the metal porous material is between 50-70%, wherein the metal porous material has a decomposition point of between 150-250° C.

9. A method for detecting nitrogen-containing compounds, comprising:
providing the metal porous material as claimed in claim 1;
introducing a gas sample to react with the metal porous material; and
analyzing results of the reaction.

10. The method as claimed in claim 9, wherein the nitrogen-containing compounds comprise ammonia gas.

11. The method as claimed in claim 9, further comprising:
connecting the metal porous material with a UV-Visible spectroscopy system for real-time detection of absorption intensity within a specific wavelength range of the metal porous material.

12. The method as claimed in claim 11, wherein the specific wavelength range is between 300-900 nm.
